(19) United States
(12) Patent Application Publication
(10) Pub. No.: US 2018/0349821 A1
(43) Pub. Date: Dec. 6, 2018

(54) WORKFORCE SCHEDULING

(71) Applicant: KONINKLIJKE PHILIPS N.V., EINDHOVEN (NL)

(72) Inventors: Xuanqi ZHANG, Boston, MA (US); Jingyu ZHANG, Elmsford, NY (US); Xiang ZHONG, Cambridge, MA (US)

(21) Appl. No.: 15/779,182

(22) PCT Filed: Nov. 7, 2016
(86) PCT No.: PCT/IB2016/056672

§ 371 Date: May 25, 2018

Related U.S. Application Data
(60) Provisional application No. 62/260,738, filed on Nov. 30, 2015.

(51) Int. Cl. G06Q 10/06; G16H 40/20; G06N 7/00

(52) U.S. Cl. CPC G06Q 10/06311; G16H 40/20; G06N 7/005; G06Q 10/0633; G06Q 10/06312

(57) ABSTRACT

A method includes receiving, in electronic format, a set of predetermined workforce related inputs, generating, using a stochastic model, a core-staff assignment, a float-pool assignment, and an overtime and agency cover for a hospital unit based on the set of predetermined inputs, generating a core-staff workings pattern and a float-pool working pattern for the hospital unit based on the core-staff assignment, the float-pool assignment, and a predetermined set of schedule rules, and employing the core-staff workings pattern and a float-pool working pattern to construct a workforce schedule in the electronic format for the hospital unit. Generally, the approach herein can consider from budget level to implementation level for a hospital in workforce scheduling, includes solutions for nurse absence and high-low patient census situations, and can accommodate different shift start time and/or shift length designs for both optimization and implementation purpose.

### FIELD OF THE INVENTION

The following generally relates to workforce scheduling and is described with particular application to hospital nurse scheduling. However, it is also amenable to other hospital staff scheduling and/or non-hospital staff scheduling.

### BACKGROUND OF THE INVENTION

Staffing is a cost for hospitals, and aligning the staffing level with patient demand variation can be challenging. Hospitals have faced the dilemma of saving operating cost and maintaining a satisfactory patient to nurse ratio. However, sometimes there are more nurses scheduled than needed in a unit. This results in cost and staffing inefficiencies. Other times, e.g., to cover an unexpected increase in patient volume where there is not enough nurses, nurses (e.g., via overtime, agency, etc.) are added to make up for the deficiency. In this instance, the initial patient to nurse ratio may not meet the satisfactory patient to nurse ratio. In general, there is no workforce staffing golden rule. Instead, the staffing level and scheduling process depends on a nurse manager's personal experience and manual arrangement. Current scheduling software does not utilize an optimal combined resource for workforce or accommodate schedule pattern accordingly. Thus, there is an unresolved need for another approach to improve workforce scheduling.

### SUMMARY OF THE INVENTION

Aspects of the present application address the above-referenced matters and others.

According to one aspect, a method includes receiving, in electronic format, a set of predetermined inputs, generating, using a stochastic model, a core-staff assignment, a float-pool assignment, and an overtime and agency cover for a hospital unit based on the set of predetermined inputs, generating a core-staff workings pattern and a float-pool working pattern for the hospital unit based on the core-staff assignment, the float-pool assignment, and a predetermined set of schedule rules, and employing the core-staff workings pattern and a float-pool working pattern to construct a workforce schedule in the electronic format for the hospital unit.

In another aspect, a computing system includes a memory device configured to store instructions, including a record integration module, and processor configured to executes the instructions. The processor, in response to executing the instructions: receives, in electronic format, a set of predetermined inputs, generates, using a stochastic model, a core-staff assignment, a float-pool assignment, and an overtime and agency cover for a hospital unit based on the set of predetermined inputs, generates a core-staff workings pattern and a float-pool working pattern for the hospital unit based on the core-staff assignment, the float-pool assignment, and a predetermined set of schedule rules, and employs the core-staff workings pattern and a float-pool working pattern to construct a workforce schedule in the electronic format for the hospital unit.

In another aspect, a non-transitory computer readable medium is encoded with computer executable instructions, which, when executed by a processor of a computer, cause the computer to: receive, in electronic format, a set of predetermined inputs, generate, using a stochastic model, a core-staff assignment, a float-pool assignment, and an overtime and agency cover for a hospital unit based on the set of predetermined inputs, generate a patient-to-nurse ratio distribution and a resource utilization for the hospital unit based on the core-staff assignment, the float-pool assignment, and the overtime and agency cover, generate a core-staff workings pattern and a float-pool working pattern for the hospital unit based on the core-staff assignment, the float-pool assignment, and a predetermined set of schedule rules, and employ the core-staff workings pattern and a float-pool working pattern to construct a workforce schedule in the electronic format for the hospital unit.

Still further aspects of the present invention will be appreciated to those of ordinary skill in the art upon reading and understand the following detailed description.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an example system with workforce and scheduling optimization modules.

FIG. 2 schematically illustrates an example system of the workforce optimization module.

FIG. 3 schematically illustrates an example system of the scheduling optimization module.

FIG. 4 shows an example of average hourly census for one unit.

FIG. 5 shows an example of a hospital's general information for each unit.

FIG. 6 shows an example of desired maximal PTN ratios for each unit in different shifts.

FIG. 7 shows an example of the core-staff cost, float-pool needs and total cost for each unit.

FIG. 8 shows an example of core-staff assignment, float-pool assignment and overtime and agency cover.

FIG. 9 shows an example of a first-week working pattern for one unit.

FIG. 10 shows an example of a second-week working pattern for one unit.

FIG. 11 shows an example of a third-week working pattern for one unit.

FIG. 12 shows an example of a fourth-week working pattern for one unit.

FIG. 13 illustrates an example method according to an embodiment herein.

### DETAILED DESCRIPTION OF EMBODIMENTS

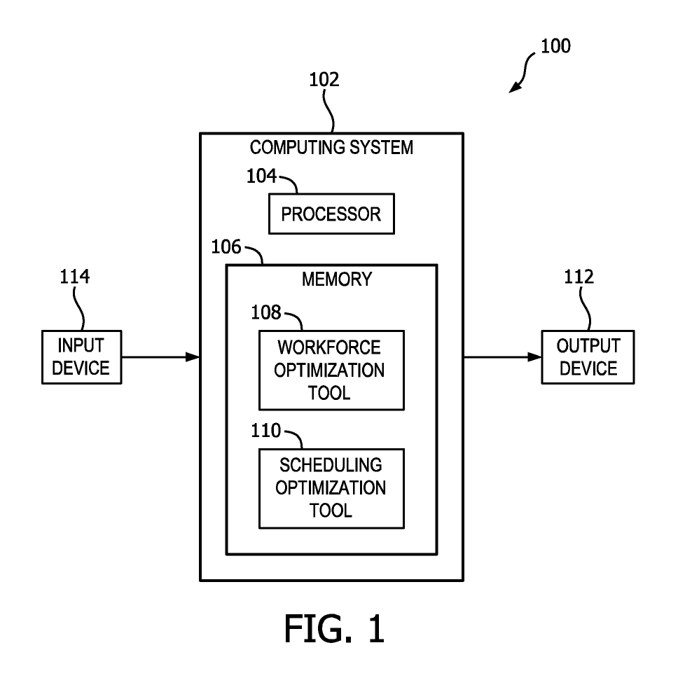

FIG. 1 illustrates a system 100. The system 100 includes a computing system 102. The computing system 102 includes at least one processor 104 (e.g., a microprocessor, a central processing unit, etc.) that executes at least one computer readable instruction stored in computer readable storage medium (“memory”) 106, which excludes transitory medium and includes physical memory and/or other non-transitory medium. The at least one computer readable instruction, in this example, includes a workforce optimization tool 108 with corresponding instructions and a scheduling optimization tool 110 with corresponding instructions. The system 100 further includes an output device(s) 112 such as a display monitor, portable memory, a network interface, etc. The system 100 further includes an input device(s) 114 such as a mouse, keyboard, a network interface, etc.

Generally, the instructions of the workforce optimization tool 108, when executed by the at least one processor 104, cause the at least one processor 104 to acquire historical hourly census of each unit in a hospital and process this data to create hourly patient arrival probability distributions, and then simulate a random patients' arrival based on this information using a stochastic workforce optimization model. Based on the incoming patients, the model computes the optimal core-staff, float-pool, overtime and agency staffing level at any hour, shift, day, week or season. These results reflect actual scenarios in a hospital. The core-staff assignment determines how the core-staff fulfills the schedule, and the float-pool, overtime and agency staffing level determine float-pool and overtime and agency on-demand arrangement. This information, e.g., is used to generate an actual patient-to-nurse (PTN) ratio distribution and a resource utilization level. The generated actual patient-to nurse ratio distribution is different from an input desired patient to nurse ratio, and can reflects if the desired ratio has been achieved and/or indicates the actual staffing situation, understaffed or overstaffed.

The instructions of the scheduling optimization tool 110, when executed by the at least one processor 104, cause the at least one processor 104 to process the core-staff assignment and float-pool assignment from the workforce optimization tool 108 along with scheduling rules to create a core-staff and float-pool working patterns. The working patterns indicate the number of staff needed on each shift on each day of the week for every hospital unit and a specific arrangement to each tentative roaster. The core-staff working patterns are made available for core-staff nurses from each unit to pick according to work rules, weekends worked in the past, and their seniorities. The remainders (position vacancies due to nurse turnover, absence due to family medical leave act (FMLA) and part of the non-productivity related absence) become holes in the overall schedule and will be added to float-pool assignment and covered by shared float-pool nurse.

In one instance, the workforce optimization tool 108 and/or the scheduling optimization tool 110: (1) reduce staffing cost and avoid understaff in changing patients demand by using simulation-based stochastic optimization in workforce optimization tool; (2) generate working patterns based on optimal staffing level so every nurse could choose their patterns at the beginning of the scheduling cycle; (3) adapt different hospitals' requirements by having flexible and/or optimized shift design (the traditional 12 hour per shift results, 12 plus 8 hour combined shift, etc.); and (4) generate expected results under optimal workforce strategy to have the actual resource status like patient to nurse (PTN) ratio distribution. Furthermore, the tools 108 and/or 110 can optimize the workforce level further using flexible shift starting time and/or flexible shift length (4 hours, 8 hours, and 12 hours). This can save the total FTE amount, especially for units with large patient census variations. It is also suitable for hospitals with different starting time and shift length requirements.

Furthermore, the tools 108 and 110 described herein require less memory and/or faster computation time, while improving scheduling results, relative to other scheduling tools.

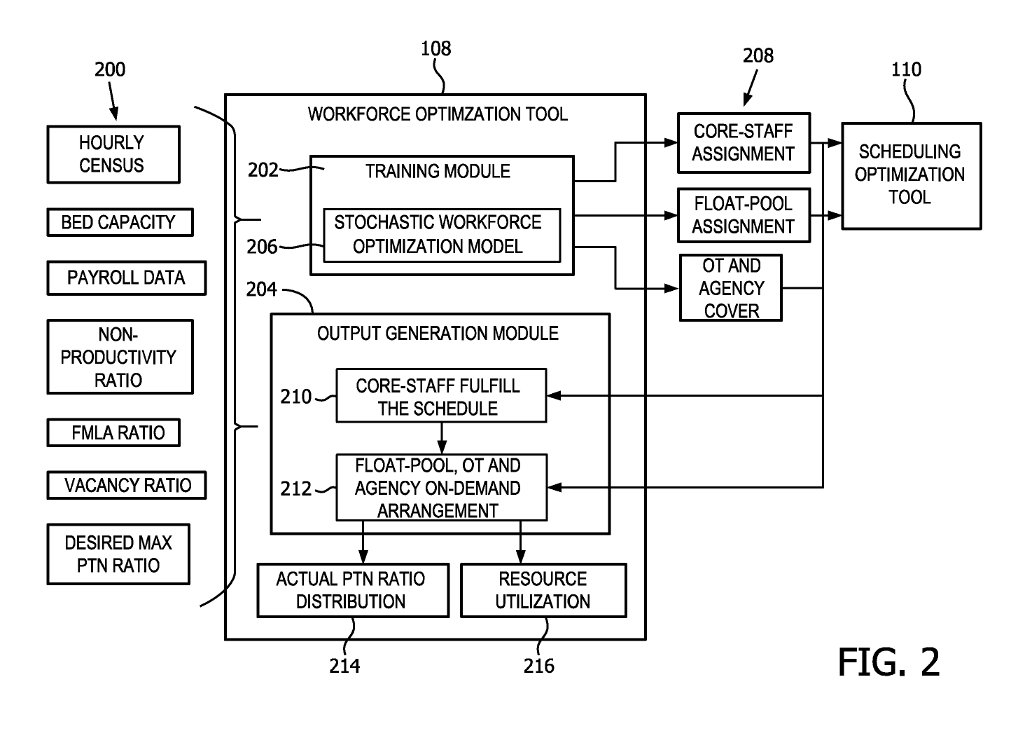

FIG. 2 schematically illustrates an example of the workforce optimization tool 108 in connection with the scheduling optimization tool 110. In this example, the workforce optimization tool 108 includes a training mode module 202 and an output generation module 204.

In this example, the training and output generation modules 202 and 204 receive, as input 200, one or more of the following, and/or additional and/or alternative data:
Hourly census—For example, an average and/or a standard deviation of historical data, which includes a number of patients that occupied beds in each hour in each hospital unit;
Bed capacity—For example, a total number of beds set up for each hospital unit to use;
Payroll data—For example, a weighted average hourly wage for each unit's core-staff, weighted average hourly wage for float-pool and agency nurse;
Non-productivity ratio—For example, an average non-technique activity over annual working time in percentage for each hospital unit, activities include: training, orientation etc.;
FMLA ratio—For example, an average family medical leave time over annual working time in percentage for each hospital unit;
Vacancy ratio—For example, a sum of working hours assigned to vacant positions over total working hours in percentage for each unit, and
Desired maximum patient-to-nurse (PTN) ratio—For example, a number of patients assigned to each nurse.

The modules 202 and 204 generate, as output 208, 214 and 216, one or more of the following, and/or additional and/or alternative data:
Core-staff Assignment—For example, the number of employees needed for each shift weekly;
Float-pool Assignment—For example, a percentage of chances a float pool nurse will be needed weekly in each shift;
Overtime and Agency Cover—For example, unexpected work load covered by core-staff;
Actual PTN Ratio distribution—For example, the actual PTN ratio trend by applying the optimal training results, and
Resource utilization—For example, this is used to evaluate the real resource usage from the output generation module.

The training module 202 employs a stochastic workforce optimization model 206 (which is described in detail below) to determine an optimal combination of staffing levels such as the number needed for each shift for a day for core-staff, float-pool nurse, overtime nurse and agent nurse. Core-staff are responsible for the major level of patients, while the float pool, overtime nurse and agency nurse should cover patient volume variation and core-staff absences. A float-pool nurse is a relatively cheaper and flexible resource, which is shared by several related hospital units where similar skill sets are required. An overtime nurse is a core-staff nurse working overtime hours. The overtime wage, in one example, is 1.5 times the non-overtime wage. A float-pool nurse's wage is less than 1.5 times, but higher than regular core-staff wage. An agency nurse is the most expensive resource, but may be needed for unexpected vacancies. The stochastic workforce optimization model 206, in one instance, assigns resources based on their wages to save total operating cost.

The output generation module 204 processes the output of the training module 202 and generates the actual PTN ratio distribution 214 and the resource utilization 216, and/or other performance factors. The number of core-staff nurse on duty on each shift on the day of week is based on the core-staff assignment, and the number of float-pool nurse on duty on each shift on the day of week is based on float-pool assignment. However, the real number of float-pool nurse, which has been assigned to every unit changes according to patient volume variation. Therefore assigning a float pool nurse to each unit is designed to satisfy those high unexpected patients' volume units first, which may cause different levels of understaff for other units or overall overstaff sometimes. The output generation module 204 simulates the working space under optimal strategy and would reflect more realistic PTN ratios distribution and resource utilization level.

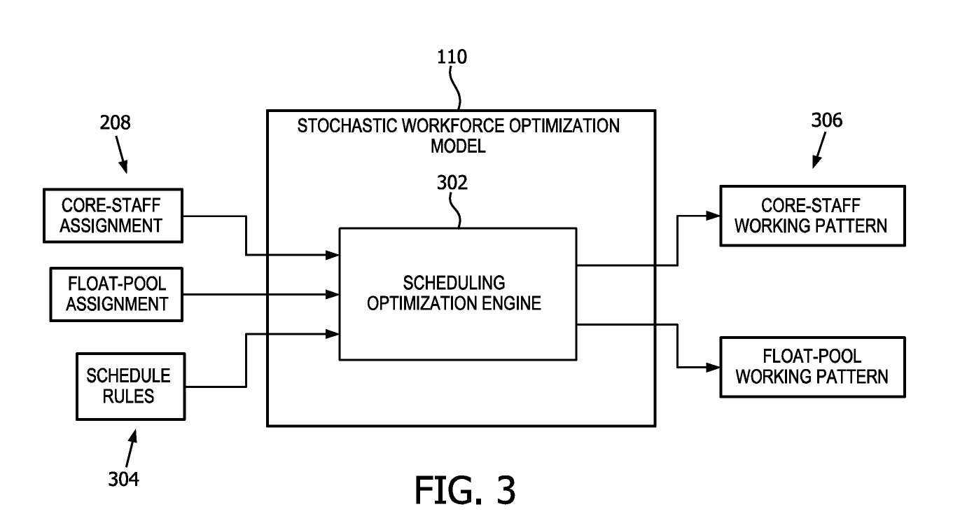

FIG. 3 schematically illustrates an example of the scheduling optimization tool 110. The scheduling optimization tool 110 includes a scheduling optimization engine 302. The scheduling engine 302 receives, as input, the core-staff and float-pool assignments 208 generated by the training module 202 and schedule rules 304. Examples of the schedule rules 304 include a maximum consecutive working days per week, total working hours per week, single weekend work rule, consecutive weekend working rules, the minimum rest time between shifts, etc. The scheduling engine 302 generates, as output 304, one or more of the following and/or additional or alternative data:
Core-staff working pattern. For example, the number of core-staff needed and the working rules. This indicates the working days arrangement for core-staff, and
Float-pool working pattern. For example, this represents the shared pool's float nurses' working pattern.
   The working patterns 306 provide nurses a specific working date in a period working cycle (the scheduling cycle can be flexible to any two-week cycle extension) without violating any of the (working and rest) schedule rules 304.

By way of example, in the beginning of the scheduling cycle, core-staff first picks the working dates to cover the majority of the demands, and they pick according to their seniority level, depending on the rules and conventions for each unit. Then, a portion of the known absent working dates will be added to the float-pool working pattern and will be covered by float-pool. The other part will be covered by core-staff themselves. The remainder will be covered by over-time or agency nurse. The float-pool working pattern is from the needed department by float-pool assignment. The float-pool assignment is to cover patient variations in iterations for each unit, which becomes the first part of float-pool use. Based on other historical absence ratios like FMLA ratio, other part of float-pool use is predicted for the coming year. The total float-pool needs from the shared units are determined by adding them together and used to generate float-pool working patterns.

From above, the training module 202 employs the stochastic workforce optimization model 206 to determine an optimal combination of staffing levels, including the number needed for each shift for a day for core-staff, float-pool nurse, overtime nurse and agent nurse. The following provides a non-limiting example of the stochastic workforce optimization model 206.

Indices and sets: N1 set of staff types (RN, PCT); i ∈ N
S set of shifts; j ∈ S
T time slots (e.g., can have a unit of 1 hour); t ∈ T
S(t) shifts that contain time slot t
W set of patient arrival scenarios (according to the patient arrival rate, different scenario will be randomly generated based on iteration); w ∈ W

Parameters:
cᵢ cost per FTE for staff type i
fⱼ number of FTE needed for shift j (1.5 for 12-hour shift, 1 for 8-hour shift, 0.5 for 4-hour shift)
rᵢₜ maximum patient to staff ratio for staff i in time slot t
TNᵢ total FTE number of staff type i
q_{w} probability of patient arrival scenarios w
p_{tw} patient volume at time slot t in scenarios w
Fᵢ float nurse i's maximum FTE
Aᵢ maximum number of agency nurse i available
α₁ weight for total FTE cost
α₂ weight for fulltime employees' overtime FTE cost
α₃ weight for float employees' FTE cost
α₄ weight for agency employee's FTE cost
p₁ average proportion of non-productive hours in total effective working hours
p₂ average proportion of FMLA days in total working days—5%
p₃ average proportion of vacant FTEs in total FTEs
   u core-staff cover non-productivity percentage

Decision variables:
Xⱼⱼ number of nurses of type i assigned to shift j
p_{ijw} number of type i overtime nurses needed at shift j in scenario w
y_{ijw} number of additional type i float nurses needed at shift j in scenario w
β_{ijw} number of additional type i agency nurses needed at shift j in scenario w
xᵢⱼ, z_{itw}, y_{ijw}, β_{ijw}, Fᵢ, Aᵢ nonnegative integer, ∀i ∈ N, j ∈ S, t ∈ T, w ∈ W.

Mathematical formulation: Minimizeα₁Σ_{i,j}cᵢxᵢⱼ(1+u*p₁)+α₂Σ_{j,w}q_{w}cᵢz_{ijw}+α₃(Fᵢ+Σ_{i,j}cᵢx_{i,j}((1−u)p₁+p₂))+(α₃−α₁)Σ_{i,j}cᵢx_{i,j}p₃+α₄Σ_{j,w}q_{w}cᵢβ_{ijw}. Regular nurse FTE cost+Extra nurse FTE cost (includes fulltime nurses' overtime, budgeted float nurses and agency nurses, α₁=1, and α₁<α₃<α₂<α₄. Where the above is subject to:
1) Total FTE number of nurse type i: Σⱼfⱼ(xᵢⱼ+Σ_{w}q_{w}×(y_{ijw}+β_{ijw}))≤TNᵢ, ∀i ∈ N. For nurse type i, the needed FTE<=Total available FTE.
2) Maximum patient to nurse ratio: rᵢₜ(Σ_{j ∈ S(t)}xᵢⱼ+z_{ijw}+y_{ijw}+β_{ijw})≥p_{tw}, ∀i ∈ N, t ∈ T, w ∈ W. Patient volume baseline+Extra patient volume under any iteration>=patient volume at any iteration and any slot, and
3) Maximum float nurses' FTE: Σ_{j,w}q_{w}cᵢy_{ijw}y_{ijw}≤Fᵢ, β_{ijw}≤Aᵢ, ∀i ∈ N, j ∈ S, w ∈ W. Assigned float nurses total's FTE is less than the budgeted float pool FTE over long run. Assigned agency nurses are less than the available nurses.

In one instance, the objective of the stochastic workforce optimization model is to save cost and arrange float-pool effectively. The total cost equals the summation of core-staff regular time cost, core-staff overtime cost, budgeted float-pool cost and the agency nurse cost in FTE terms. A heuristic approach is applied in the model to search for the combination of the optimal number of core-staff nurse, float-pool nurse, overtime nurse and agency nurse.

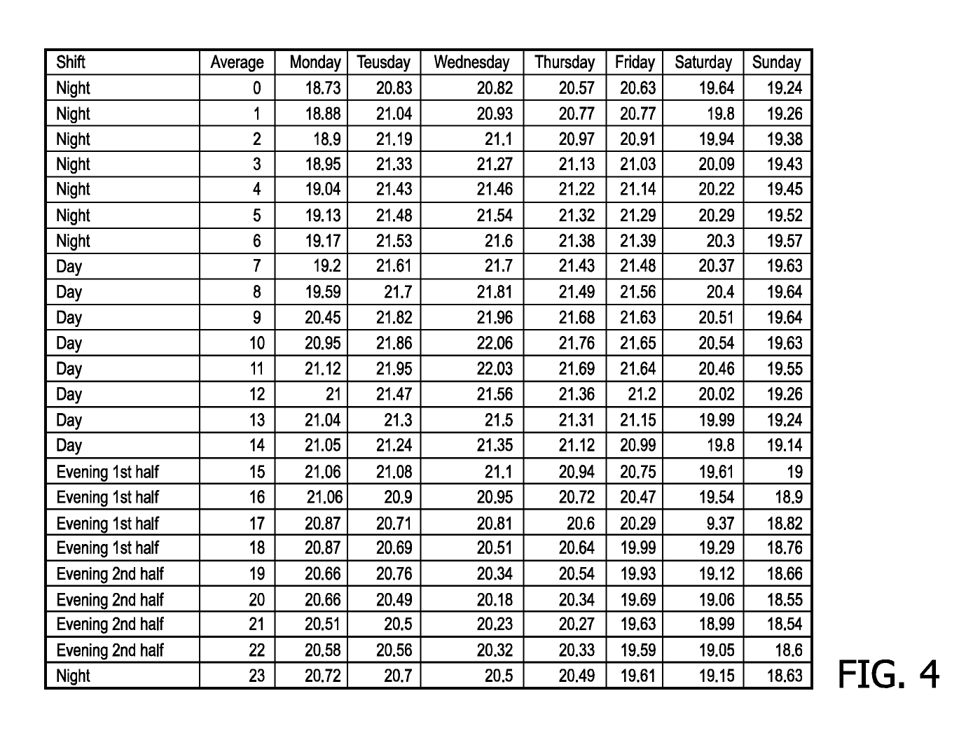

The following provides a non-limiting use-case in connection with FIGS. 4-9.

In this example, the input 200 is from human resources (HR) and operational data. Historical hospital hourly census data is converted into two datasets: 1) average hourly census and 2) standard deviation hourly census so it can follow a normal distribution. FIG. 4 shows average hourly census for one unit. A corresponding table for standard deviation is in the same format. In FIG. 4, a first column shows the shift names, and a next column gives the hours in that shift accordingly. Different shift designs can be used across hospitals. The random patient arrivals of every hour of the day of the week is generated according to the mean and standard deviation of hourly census data of a unit. This data could be obtained from arrival discharge data (ADT) data and transfer log (available from a hospital IT system).

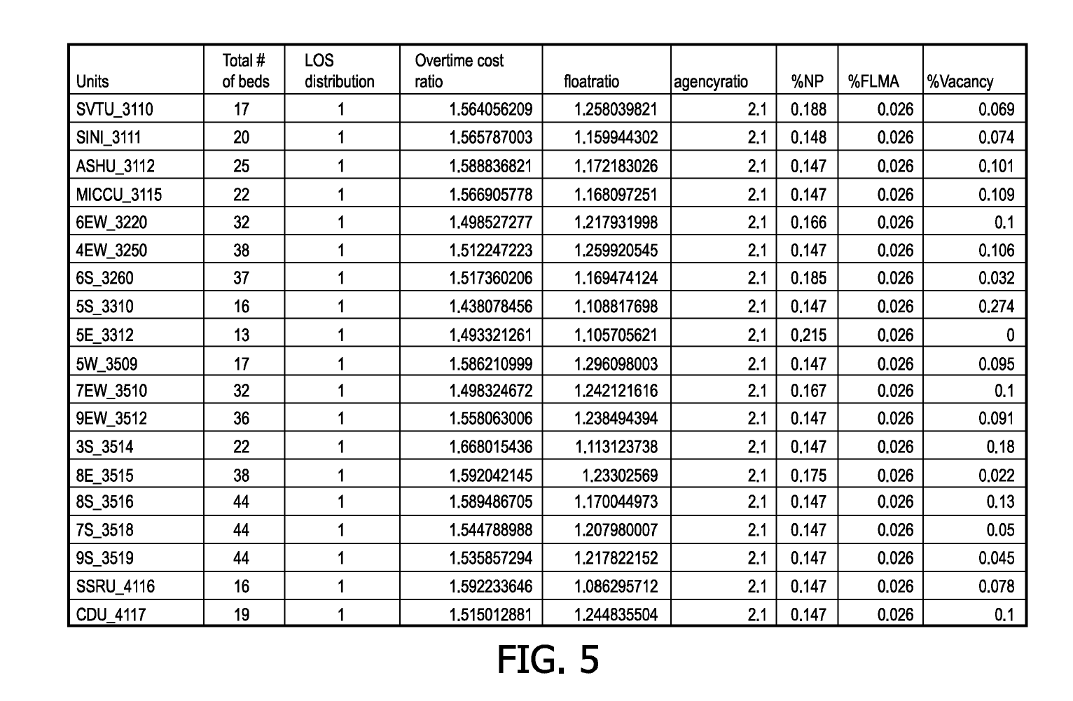

FIG. 5 shows hospital's general information for each unit including bed capacity, overtime cost ratio, float-pool ratio, agency ratio, non-productivity ratios (NP), FMLA ratio and vacancy ratio. Bed capacity is a number which could be used to check hourly census. The number generated for patient arrivals is capped by bed capacity. Payroll data determines the combination of different resources. In the model, the relative salary ratio over the core-staff full-time-equivalent (FTE) is used in order to compute the total cost in terms of core-staff FTE. However, other forms of costs (such wage salary in any currency) can also be used. The average core-staff wage is set to be one (1) in this example as the base wage, and overtime cost ratio, float ratio and the agency ratio are ratios between overtime wage, float-pool wage, agency wage and base wage. Non productivity, FMLA, and Vacancy ratios are all about core-staff's absence, which would decide the annual float-pool usage for specific hospital unit. FMLA and vacancy absences are usually fully covered by float-pool nurse while a proportion of non-productivity will be covered by float-pool and the remainder will be covered by core-staff themselves.

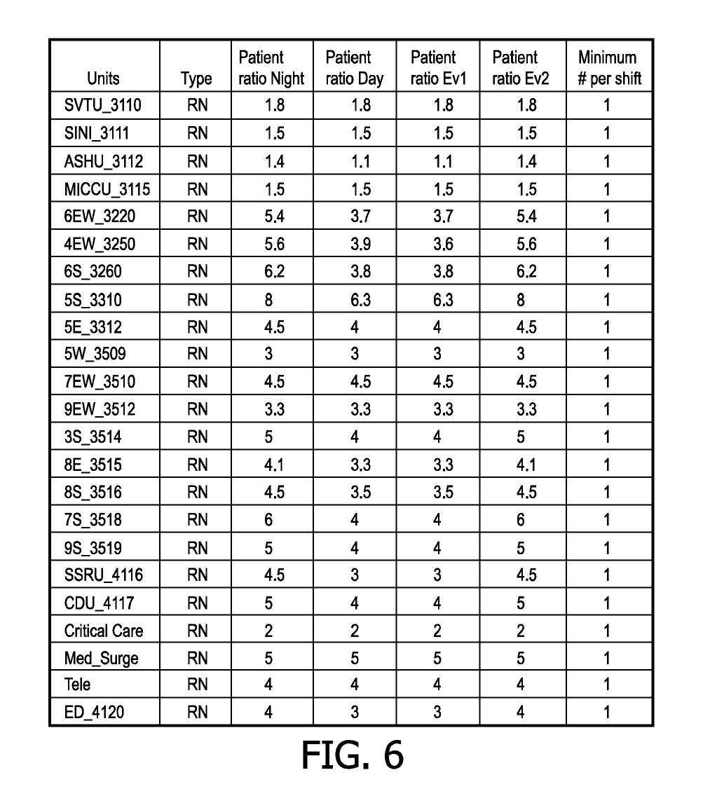

FIG. 6 shows desired maximal PTN ratios for each unit in different shifts. RN indicates registered nurse. The last column is a minimum RN requirement for each unit.

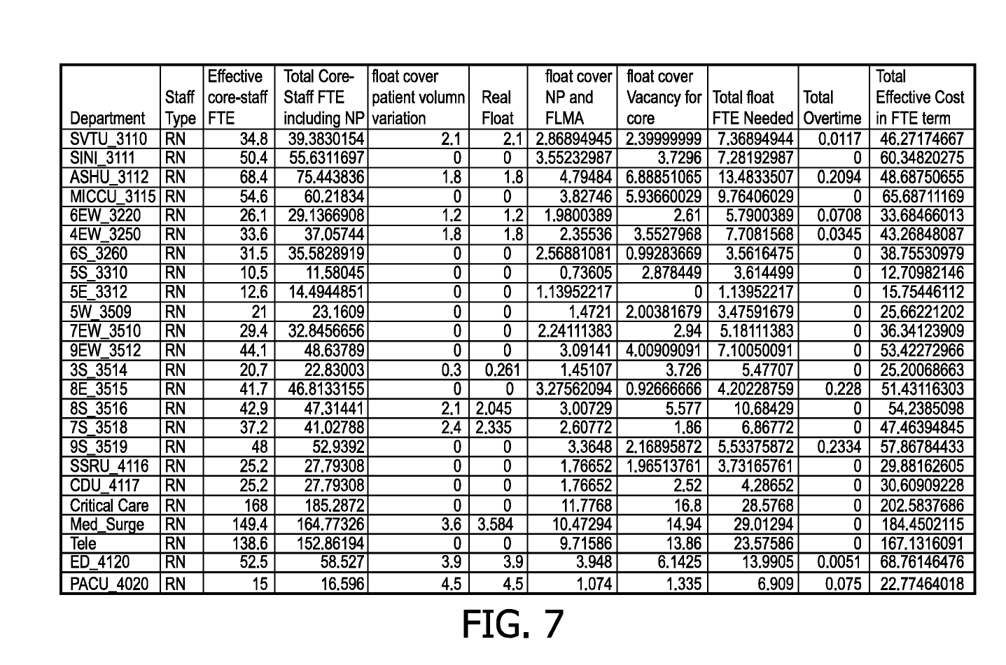

FIG. 7 shows the core-staff cost, float-pool needs and total cost for each unit. This optimal float-pool nurse cost only stands for float-pool covers patient census variation, and these will be reflected in the model's general information results.

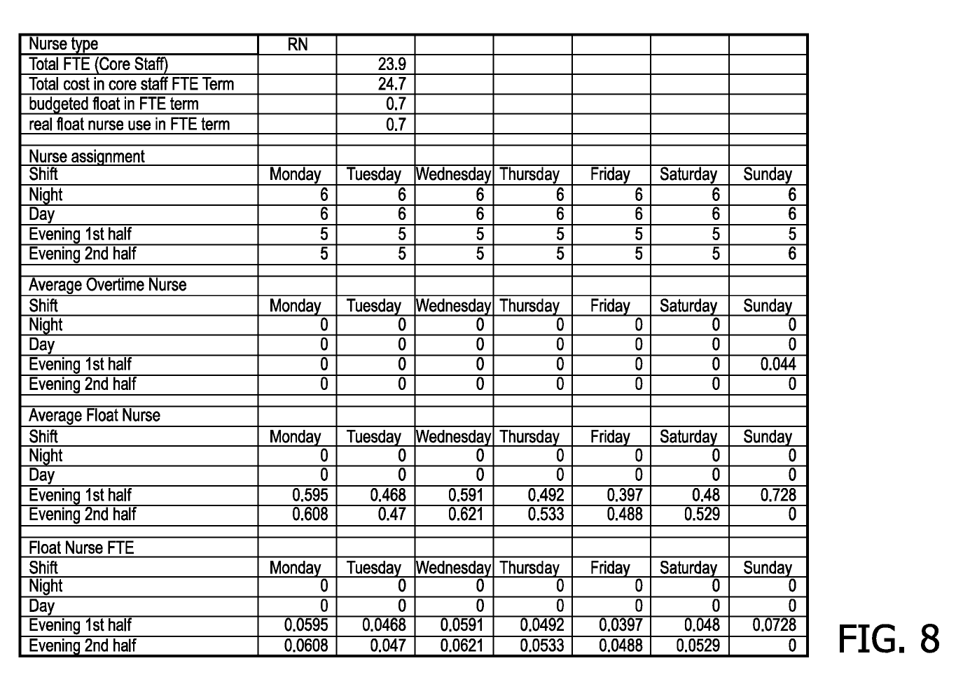

FIG. 8 shows core-staff assignment, float-pool assignment and overtime and agency cover. In this example, because of the cost factor and relaxed size of float-pool nurse, the result avoids the agency usage, and there is no agency use information from this table. From the “Nurse assignment” section, the optimal core-staff number in each shift from the day of the week has been captured. According to the optimal core-staff number in each shift and the shift length, total core-staff in FTE term is listed in the second row. However, the float-pool nurse assignment means usage frequent in certain amount of iterations under the above core-staff on duty's condition, which is in the fourth section. For example, on Monday's first evening shift, under the optimal core staff level, which equals 5, in 10000 iterations, 595 times one float-pool nurse is needed.

The core-staff assignment and the float-pool assignment 208 are input to the scheduling optimization tool 110, along with the schedule rules 304, which must be satisfied. Example schedule rules 304 include the following; 1) maximum consecutive working days should be less than or equal to four; 2) maximum working hours per week should be less than or equal to forty; 3) day shift nurse can only work day shift, night shift nurse can only work night shi; 4) during weekends, one nurse should take all the weekends shifts for one week, and take the all the second weekend shifts off, etc. Other soft rules may also apply including: use less part-time nurse as possible, reduce the inequality in schedules by rotating patterns and so on.

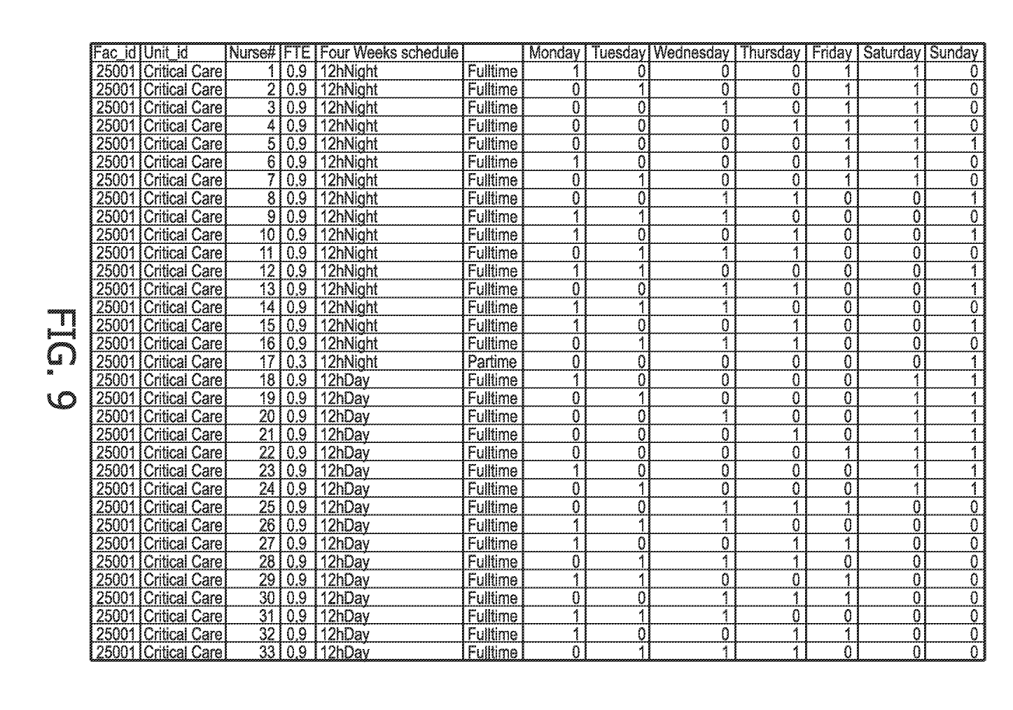

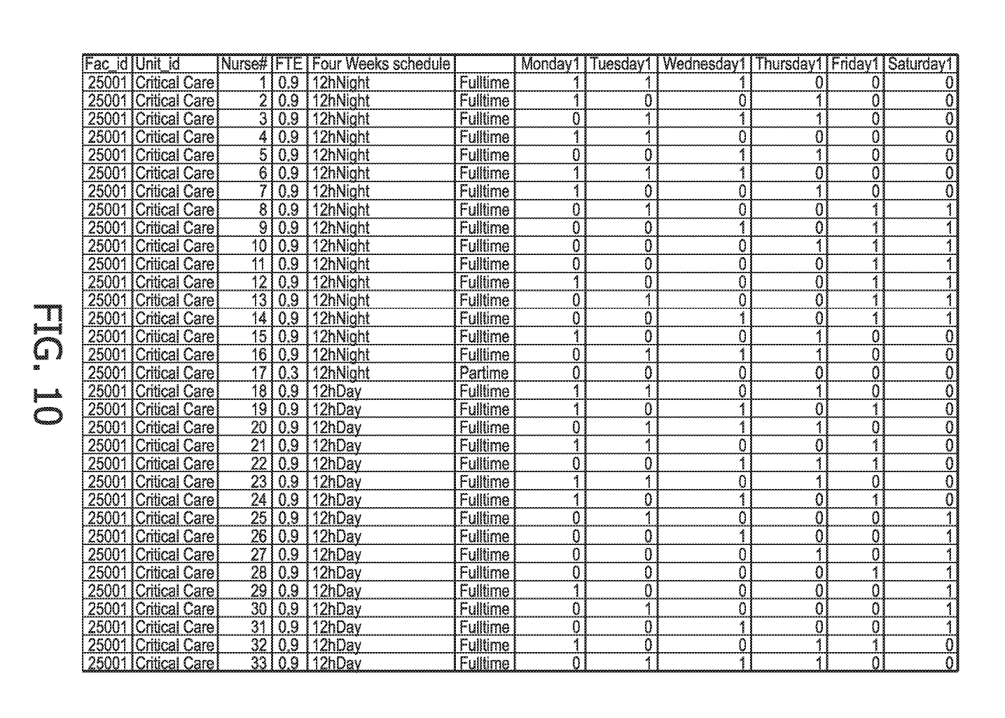

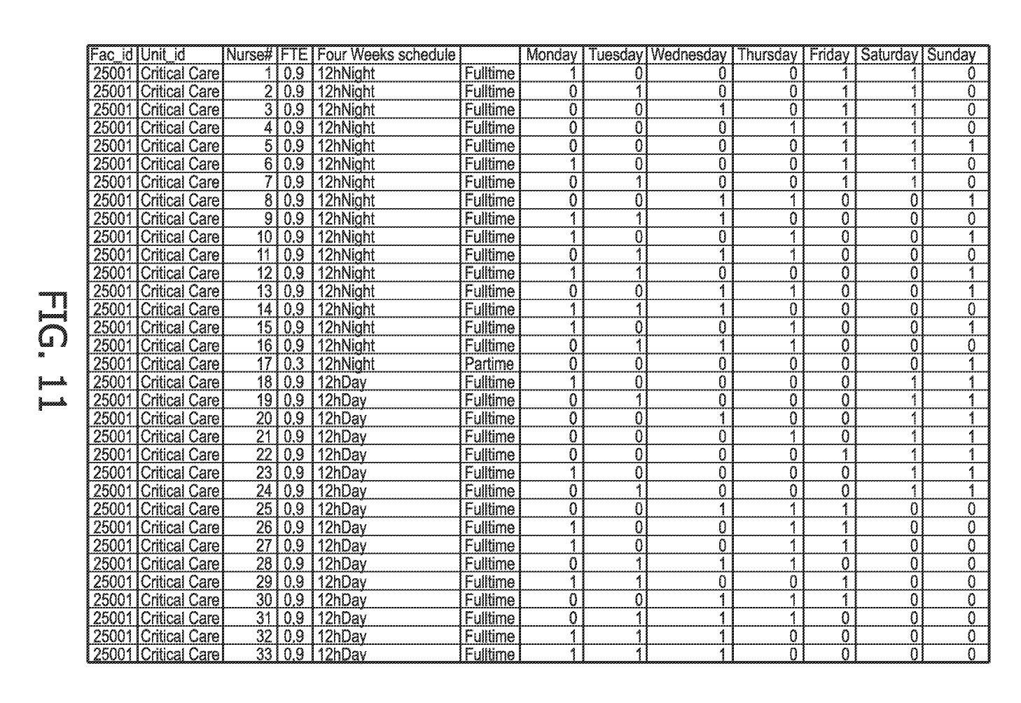

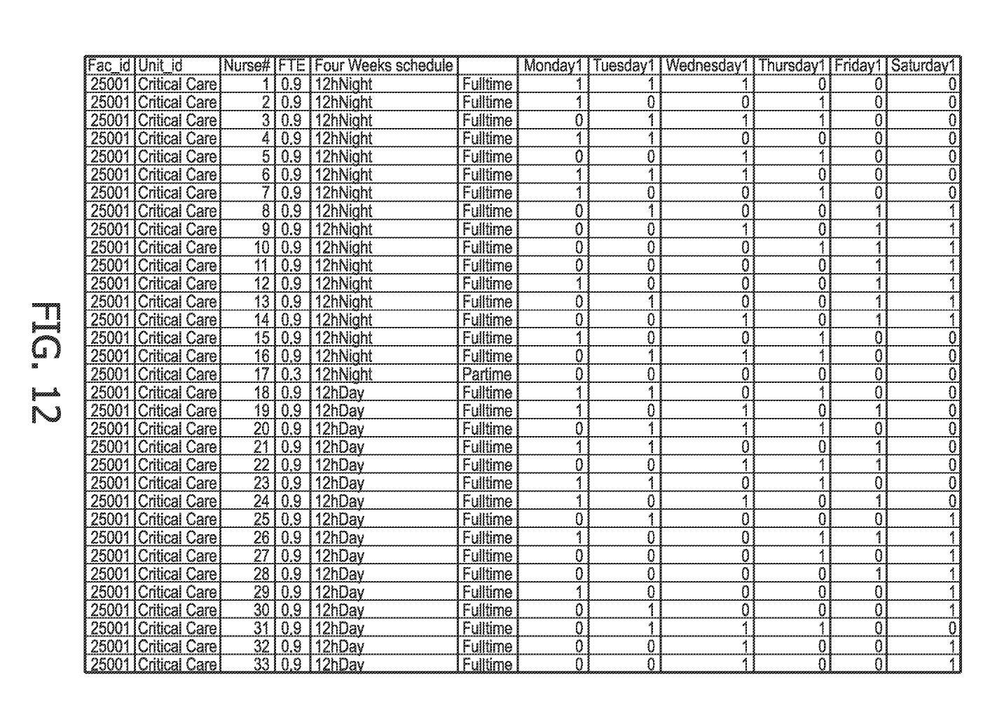

FIGS. 9, 10, 11 and 12 show an example of a four-week working pattern for one unit, which follows the rules 304. FIG. 9 shows a pattern for a first week, FIG. 10 shows a pattern for a second week, FIG. 11 shows a pattern for a third week, and FIG. 12 shows a pattern for a fourth week. The first column in the figures indicates the hospital name. The second column indicates the unit name. The third column indicates the nurse number. The fourth column indicates FTE for this nurse. This example is the working pattern for a 12 hour shift. It has night and day shift working patterns. A weekend is defined as Friday, Saturday night, and Saturday and Sunday day shift. The following steps are used to populate the cells: 1) make the weekends blocked first; 2) fill up the weekday tables by the schedule rules; and 3) make possible full time nurse first and the remainders become part-time nurse.

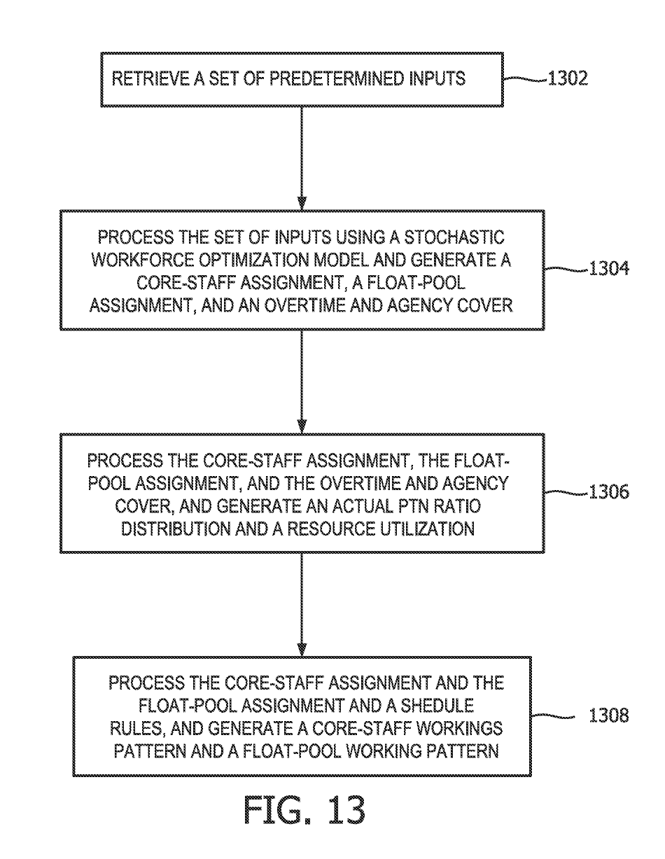

FIG. 13 illustrates an example method according to an embodiment herein.

It is to be appreciated that the ordering of the acts in the methods described herein is not limiting. As such, other orderings are contemplated herein. In addition, one or more acts may be omitted and/or one or more additional acts may be included.

At 1302, a set of predetermined inputs are retrieved, as described herein and/or otherwise.

At 1304, the training module 202 processes the set of inputs using the stochastic workforce optimization model 206, and generates a core-staff assignment, a float-pool assignment, and an overtime and agency cover, as described herein and/or otherwise.

At 1306, the output generation module 204 processes the core-staff assignment, the float-pool assignment, and the overtime and agency cover, and generates an actual PTN ratio distribution and a resource utilization, as described herein and/or otherwise.

At 1308, the scheduling optimization tool 110 processes the core-staff assignment and the float-pool assignment and a schedule rules, and generates a core-staff workings pattern and a float-pool working pattern, as described herein and/or otherwise.

The above may be implemented by way of computer readable instructions, which when executed by a computer processor(s), cause the processor(s) to carry out the described acts. In such a case, the instructions can be stored in a computer readable storage medium associated with or otherwise accessible to the relevant computer. Additionally or alternatively, one or more of the instructions can be carried by a carrier wave or signal.

The tools described herein quantitatively determine float pool size, unit-specific budget, and arrangement towards each shift and unit, as well as the optimal staffing mix of core staff, float pool, core staff overtime, and agency nurse. The float pool alleviates the core staff overtime usage during unexpected surge in patient volume. The non-productivity, vacancy and Family and Medical Leave Act (FMLA) absence ratio of core staffs have been also considered in calculating the optimal results to have these occasions covered in plan. The tools can optimally schedule nurses to fulfill the required staffing levels for different nurse types and can show how the shift designs change will result in operating cost change is able to be compared for the hospital. One example shows that 12 hour plus 8 hour shift combination results overall have lower cost than the traditional 12 hour shift.

The integration between workforce and scheduling optimization: workforce model results could show the optimal number of each resource in each shift for different departments, and based on which the working patterns could be generated. The model can be adjusted to accommodate any scheduling rules required by hospital administrator and detailed working patterns are made available (e.g. Full-time Nurse A should work on Monday, Tuesday and Thursday's 12 hour day shift for week 1) for each staff in every unit in a scheduling cycle. In summary, the solution mechanism, which includes analyzing the optimal workforce and generating working pattern accordingly, meets the hospital needs and has minimized the gap between research and practice. Stochastic simulation makes the patient variation be taken in to account, which can provide more robust and confident results.

The invention has been described herein with reference to the various embodiments. Modifications and alterations may occur to others upon reading the description herein. It is intended that the invention be construed as including all such modifications and alterations insofar as they come within the scope of the appended claims or the equivalents thereof

1. A method, comprising:
receiving, in electronic format, a set of predetermined workforce related inputs;
generating, using a stochastic model, a core-staff assignment, a float-pool assignment, and an overtime and agency cover for a hospital unit based on the set of predetermined inputs;
generating a core-staff workings pattern and a float-pool working pattern for the hospital unit based on the core-staff assignment, the float-pool assignment, and a predetermined set of schedule rules; and
employing the core-staff workings pattern and a float-pool working pattern to construct a workforce schedule in the electronic format for the hospital unit.

2. The method of claim 1, wherein the first set of predetermined inputs includes:
1) a number of patients that occupied beds in the hospital unit during a predetermined time period;
2) a total number of beds in the unit;
3) weighted average hourly wages for a core-staff, a float-pool and an agency nurse pool for the hospital unit;
4) an average non-technique activity over annual working time in percentage for the hospital unit;
5) an average family medical leave time over the annual working time in percentage for the hospital unit;
6) a sum of working hours assigned to vacant positions over total working hours in percentage for the hospital unit, and
7) a number of patients assigned to each nurse in the hospital unit.

3. The method of claim 1, wherein the core-staff assignment indicates a number of employees needed for a work shift during a predetermined work period.

4. (canceled)

5. (canceled)

6. The method of claim 1, further comprising:
generating a patient-to-nurse ratio distribution and a resource utilization for the hospital unit based on the core-staff assignment, the float-pool assignment, and the overtime and agency cover.

7. (canceled)

8. (canceled)

9. The method of claim 1, wherein using the stochastic model includes minimizing the following: α₁Σ_{i,j}cᵢxᵢⱼ(1+u*p₁)+α₂Σ_{jw}q_{w}cᵢz_{ijw}+α₃(Fᵢ+Σ_{i,j}cᵢx_{i,j}((1−u)p₁+p₂))+(α₃−α₁)Σ_{i,j}cᵢx_{i,j}p₃+α₄Σ_{j,w}q_{w}cᵢβ_{ijw}, wherein α₁ is a weight for total (full time equivalent) FTE cost, α₂ is a weight for a fulltime employees' overtime FTE cost, α₃ is a weight for a float employees' FTE cost and α₄ is a weight for an agency employee's FTE cost, ci is a cost per FTE for staff type i, xⱼⱼ is a number of nurses of the type i assigned to a shift j, u is a core-staff cover non-productivity percentage, p₁ is an average proportion of the non-productive hours in total effective working hours, q_{w} is a probability of patient arrival scenarios w, z_{ijw} is a number of the type i overtime nurses needed at the shift j in the scenario w, Fᵢ is a float nurse i's maximum FTE, p₂ is an average proportion of family medical leave act days in total working days, p₃ is an average proportion of vacant FTEs in total FTEs, and β_{ijw} is a number of additional type i agency nurses needed at the shift j in the scenario w.

10. A computing system, comprising:
a memory device configured to store instructions, including a workforce optimization toll; and
a processor that executes the instructions, which causes the processor to:
receive, in electronic format, a set of predetermined inputs;
generate, using a stochastic model, a core-staff assignment, a float-pool assignment, and an overtime and agency cover for a hospital unit based on the set of predetermined inputs;
generate a core-staff workings pattern and a float-pool working pattern for the hospital unit based on the core-staff assignment, the float-pool assignment, and a predetermined set of schedule rules; and
employ the core-staff workings pattern and a float-pool working pattern to construct a workforce schedule in the electronic format for the hospital unit.

11. The computing system of claim 10, wherein the instructions further include a scheduling optimization tool, and executing the instructions further causes the processor to: generate a patient-to-nurse ratio distribution and a resource utilization for the hospital unit based on the core-staff assignment, the float-pool assignment, and the overtime and agency cover.

12. The computing system of claim 11, wherein the first set of predetermined inputs includes:
1) a number of patients that occupied beds in the hospital unit during a predetermined time period;
2) a total number of beds in the unit;
3) weighted average hourly wages for a core-staff, a float-pool and an agency nurse pool for the hospital unit;
4) an average non-technique activity over annual working time in percentage for the hospital unit;
5) an average family medical leave time over the annual working time in percentage for the hospital unit;
6) a sum of working hours assigned to vacant positions over total working hours in percentage for the hospital unit, and
7) a number of patients assigned to each nurse in the hospital unit.

13. The computing system of claim 12, wherein the core-staff assignment indicates a number of employees needed for a work shift during a predetermined work period; the float-pool assignment indicates a percentage of chances a float pool nurse is needed during the predetermined work period, and the overtime and agency cover indicates unexpected work load covered by core-staff

14. (canceled)

15. The computing system of claim 14, wherein using the stochastic model includes minimizing the following: α₁Σ_{i,j}cᵢxᵢⱼ(1+u*p₁)+α₂Σ_{j,w}q_{w}cᵢz_{ijw}+α₃(Fᵢ+Σ_{i,j}cᵢx_{i,j}((1−u)p₁+p₂))+(α₃−α₁)Σ_{i,j}cᵢx_{i,j}p₃+α₄Σ_{j,w}q_{w}cᵢβ_{ijw}, wherein α₁ is a weight for total (full time equivalent) FTE cost, α₂ is a weight for a fulltime employees' overtime FTE cost, α₃ is a weight for a float employees' FTE cost and α₄ is a weight for an agency employee's FTE cost, ci is a cost per FTE for staff type i, xⱼⱼ is a number of nurses of the type i assigned to a shift j, u is a core-staff cover non-productivity percentage, p₁ is an average proportion of the non-productive hours in total effective working hours, q_{w} is a probability of patient arrival scenarios w, z_{ijw} is a number of the type i overtime nurses needed at the shift j in the scenario w, Fᵢ is a float nurse i's maximum FTE, p₂ is an average proportion of family medical leave act days in total working days, p₃ is an average proportion of vacant FTEs in total FTEs, and β_{ijw} is a number of additional type i agency nurses needed at the shift j in the scenario w.

16. The computing system of claim 15, wherein the minimizing is subject to the following: Σ₁fⱼ(xᵢⱼ+Σ_{w}q_{w}×(y_{ijw}+β_{ijw}))≤TNᵢ, ∀i ∈ N, wherein fⱼ is a number of FTEs needed for the shift j, y_{ijw} is a number of additional type i float nurses needed at the shift j in the scenario w, TNᵢ is a total FTE number of the staff type i, and N is a set of staff types.

17. The computing system of claim 16, wherein the minimizing is further subject to the following: rᵢₜ(Σ_{j ∈ S(t)}xᵢⱼ+z_{ijw}+y_{ijw}β_{ijw})≥p_{tw}, ∀i ∈ N, t ∈ T, w ∈ W, wherein rᵢₜ is a maximum patient-to-staff ratio for the staff i in a time slot t, S(t) are shifts that contain the time slot t, p_{tw} is a patient volume at the time slot t in the scenarios w, T are time slots, and W is a set of patient arrival scenarios.

18. The computing system of claim 17, wherein the minimizing is further subject to the following: Σ_{jw}q_{w}cᵢy_{ijw}i_{ijw}≤Fᵢ, β_{ijw}≤Aᵢ, ∀i ∈ N, j ∈ S, w ∈ W, wherein Aᵢ is a maximum number of agency nurse i available, and S is a set of shifts.

19. A non-transitory computer readable medium encoded with computer executable instructions, which, when executed by a processor of a computer, cause the computer to:
receive, in electronic format, a set of predetermined inputs;
generate, using a stochastic model, a core-staff assignment, a float-pool assignment, and an overtime and agency cover for a hospital unit based on the set of predetermined inputs;
generate a patient-to-nurse ratio distribution and a resource utilization for the hospital unit based on the core-staff assignment, the float-pool assignment, and the overtime and agency cover;
generate a core-staff workings pattern and a float-pool working pattern for the hospital unit based on the core-staff assignment, the float-pool assignment, and a predetermined set of schedule rules; and
employ the core-staff workings pattern and a float-pool working pattern to construct a workforce schedule in the electronic format for the hospital unit.

20. The non-transitory computer readable medium of claim 19, wherein using the stochastic model includes minimizing the following: α₁Σ_{i,j}cᵢxᵢⱼ(1+u*p₁)+α₂Σ_{j,w}q_{w}cᵢz_{ijw}+α₃(Fᵢ+Σ_{i,j}cᵢx_{i,j}((1−u)p₁+p₂))+( ₃−α₁)Σ_{i,j}cᵢx_{i,j}p₃+α₄Σ_{j,w}q_{w}cᵢβ_{ijw}, wherein α₁ is a weight for total (full time equivalent) FTE cost, α₂ is a weight for a fulltime employees' overtime FTE cost, α₃ is a weight for a float employees' FTE cost and α₄ is a weight for an agency employee's FTE cost, ci is a cost per FTE for staff type i, xⱼⱼ is a number of nurses of the type i assigned to a shift j, u is a core-staff cover non-productivity percentage, p₁ is an average proportion of the non-productive hours in total effective working hours, q_{w} is a probability of patient arrival scenarios w, z_{ijw} is a number of the type i overtime nurses needed at the shift j in the scenario w, F₁ is a float nurse i's maximum FTE, p₂ is an average proportion of family medical leave act days in total working days, p₃ is an average proportion of vacant FTEs in total FTEs, and β_{ijw} is a number of additional type i agency nurses needed at the shift j in the scenario w.
